(19) United States
(12) Patent Application Publication
(10) Pub. No.: US 2024/0350124 A1
(43) Pub. Date: Oct. 24, 2024

(54) SPECIMEN RETRIEVAL DEVICE

(71) Applicant: THE REGENTS OF THE UNIVERSITY OF MICHIGAN, Ann Arbor, MI (US)

(72) Inventors: Mark S. COHEN, Ann Arbor, MI (US); Jeffrey PLOTT, Ann Arbor, MI (US); Jennifer LEE, Ann Arbor, MI (US); Lindsay JANES, Ann Arbor, MI (US); William James MELVIN, Ann Arbor, MI (US)

(21) Appl. No.: 18/688,522

(22) PCT Filed: Aug. 31, 2022
(86) PCT No.: PCT/US2022/042121

§ 371 Date: Mar. 1, 2024

Related U.S. Application Data
(60) Provisional application No. 63/239,459, filed on Sep. 1, 2021.

(51) Int. Cl. A61B 17/00

(52) U.S. Cl. CPC A61B 17/00234; A61B 2017/00287; A61B 2017/00367; A61B 2017/00477; A61B 2017/00862; A61B 2017/00938

(57) ABSTRACT

A device including a rod with a tip and a channel formed in the tip. The device further includes a containment member including a first end and a second end opposite the first end. The second end is coupled to the tip, and the containment member includes an opening positioned at the first end. An expansion string and a compression string are coupled to the opening of the containment member. The expansion string extends through the channel in the tip.

### CROSS-REFERENCE TO RELATED APPLICATIONS

This application claims priority to U.S. Provisional Patent Application No. 63/239,459, filed on Sep. 1, 2021, the entire contents of which are incorporated herein by reference.

### BACKGROUND

During minimally invasive surgery, conventional trocars (e.g., narrow tubes) are used through incisions to support instruments that will be operating within the body. See, for example, FIG. 1. During minimally invasive surgery, specimens will commonly need to be removed from the body cavity, such as during a laparoscopic cholecystectomy (i.e., removal of the gallbladder). Once the gallbladder is separated from surrounding tissue, the gallbladder is placed in a conventional single-use bag for removal from one of the incisions. However, the gallbladder is larger than the incisions (typically 1 cm) and once the gallbladder is positioned within the conventional bag, the bag may no longer be able to pass through the incision. In other words, when a surgeon attempts to remove the gallbladder in a conventional bag, the bag and gallbladder get stuck at the abdominal wall and not fit through the laparoscopic incisions. The harder the surgeon pulls to extract the gallbladder, the more the gallbladder specimen becomes bunched in the bag bottom, which makes removing the gallbladder even harder. In addition, the bag and gallbladder may become stuck in the abdominal wall. This problem is also seen in other minimally invasive procedures, including but not limited to VATS lobectomy, laparoscopic adrenalectomy, partial gastrectomy, partial hepatectomy, small bowel resection, colonic resection, oophorectomy, or nephrectomy.

Applying a large force to conventional bags (e.g., when attempting to retrieve a large specimen through a small incision) also risks rupturing the bag and potentially seeding bacteria, bile, stones, tumor cells, etc. from the specimen. One suboptimal solution for conventional bags is to widen the incision to accommodate the specimen, but this results in more patient pain, increased rate of hernia formation. Another suboptimal solution for conventional bags is to break up the specimen into smaller pieces (e.g., morcellation), but morcellation is a time consuming and cumbersome process that destroys the integrity of the specimen for pathologic analysis.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a laparoscopic device to remove a specimen through a laparoscopic incision by compressing the specimen and elongating the specimen along a longitudinal axis and reducing cross-sectional area.

One aspect of the present disclosure provides a device including a rod with a tip and a channel formed in the tip. The device also includes a containment member including a first end and a second end opposite the first end. The second end is coupled to the tip, and the containment member includes an opening positioned at the first end. The device further includes a string coupled to the opening of the containment member. The string extends through the channel in the tip.

In some embodiments, the device further includes a sheath defining a passageway; wherein the rod is positioned at least partially within the passageway and movable with respect to the sheath; and wherein the string extends through the passageway; wherein the opening in the containment member is oriented toward the sheath.

In some embodiments, the rod includes a rod passageway, and wherein the string extends through the rod passageway, and wherein the string includes a graspable member coupled to an end of the string.

In some embodiments, the containment member is a first containment member and the device further includes a second containment member. The first containment member is positioned at least partially within the second containment member.

In some embodiments, the first containment member is a mesh sleeve.

In some embodiments, the second containment member is impermeable to fluid.

In some embodiments, the second containment member includes a third end and a fourth end opposite the third end. The second containment member includes a second opening positioned at the third end.

In some embodiments, a drawstring is coupled to the second opening; wherein the second opening at least partially closes in response to actuation of the drawstring.

In some embodiments, the string is a first string and the device further includes a second string coupled to the opening of the containment member. The second string is further coupled to a user-actuated tightening mechanism.

In some embodiments, the opening of the containment member enlarges in response to actuation of the first string, and wherein a specimen positioned within the containment member is compressed in response to actuation of the second string.

In some embodiments, the string is one of a plurality strings coupled to the opening of the containment member, wherein the opening enlarges in response to actuation of the plurality of strings.

In some embodiments, the string is a first string, and the device further includes a second string, a third string, and a fourth string; wherein the second string is coupled to the opening within a range of 90 degrees to 120 degrees from the first string, the third string is coupled to the opening within a range of 60 degrees to 90 degrees from the second string, and the fourth string is coupled to the opening within a range of 60 degrees to 90 degrees from the third string.

In some embodiments, the rod defines a longitudinal axis and the opening faces a plane that is perpendicular to the longitudinal axis.

In some embodiments, the device further includes a handle coupled to the rod, wherein the handle includes a slot configured to releasably secure the string.

One aspect of the present disclosure provides a device including a rod with a tip and a containment member including a first end and a second end opposite the first end. The second end is coupled to the tip; and wherein the containment member includes an opening positioned at the first end. The device further includes a string looped around the opening of the containment member. The opening at least partially closes in response to actuation of the string.

In some embodiments, the device further includes a user-actuated tightening mechanism and wherein the string is coupled to the user-actuated tightening mechanism. A specimen positioned within the containment member is compressed in response to actuation of the first string by the tightening mechanism.

In some embodiments, the device further includes a handle coupled to the rod, wherein the handle includes a groove that at least partially receives the user-actuated tightening mechanism.

In some embodiments, the user-actuated tightening mechanism includes a faceted circumferential surface that is rotatable relative to the groove.

In some embodiments, the device further includes a sheath defining a passageway; wherein the rod is positioned at least partially within the passageway and movable with respect to the sheath. The string extends through the passageway; and wherein the opening in the containment member is oriented toward the sheath.

In some embodiments, the rod includes a rod passageway, and wherein the string extends through the rod passageway,

In some embodiments, the containment member is a first containment member and the device further includes a second containment member. The first containment member is positioned at least partially within the second containment member.

In some embodiments, the first containment member is a mesh sleeve, and wherein the second containment member is impermeable to fluid.

In some embodiments, the second containment member includes a third end and a fourth end opposite the third end, wherein the second containment member includes a second opening positioned at the third end.

In some embodiments, a drawstring is coupled to the second opening. The second opening at least partially closes in response to actuation of the drawstring.

In some embodiments, the string is a compression string and the device further includes an expansion string coupled to the opening of the containment member. A channel is formed in the tip and the expansion string extends through the channel.

In some embodiments, the opening of the containment member enlarges in response to actuation of the expansion string, and wherein a specimen positioned within the containment member is compressed in response to actuation of the compression string.

In some embodiments, the rod defines a longitudinal axis and the opening faces a plane that is perpendicular to the longitudinal axis.

One aspect of the present disclosure provides a device including a first rod with a first rod end, a second rod with a second rod end, and a containment member including a first end and a second end opposite the first end. The first end is coupled to the first rod end of the first rod and the second end is coupled to the second rod end of the second rod. The containment member includes an opening positioned at the first end of the containment member. The second rod is movable with respect to the first rod to elongate the containment member.

In some embodiments, the device further includes a sheath with a passageway, wherein the first rod and the second rod extend through the passageway.

In some embodiments, the second rod end moves away from the first rod end to elongate the containment member.

In some embodiments, the second rod is movable with respect to the first rod to adjust a cross-sectional area of the opening.

In some embodiments, the second rod end moves toward the first rod end to increase the cross-sectional area of the opening.

In some embodiments, the containment member is flexible and includes a mesh sleeve.

In some embodiments, the second end of the containment member is closed.

In some embodiments, the device further includes a string coupled to the opening; wherein the opening is movable between an open configuration and a sealed configuration in response to actuation of the string.

In some embodiments, the device further includes a first lock movable between an unlocked position in which the first rod is movable with respect to the second rod and a locked position in which the first rod is fixed relative to the second rod.

In some embodiments, the device further includes a second lock movable between an unlocked position in which the first rod is movable with respect to a sheath and a locked position in which the first rod is fixed relative to the sheath.

In some embodiments, the first rod includes a first handle and the second rod includes a second handle.

Other aspects of the disclosure will become apparent by consideration of the detailed description and accompanying drawings.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (also “FIG.”) relating to one or more embodiments.

FIG. 1 is a perspective view of an endoscopic surgical procedure.

FIG. 2A is a specimen retrieval device in a storage configuration.

FIG. 2B is the specimen retrieval device of FIG. 2A in a deployed configuration.

FIG. 2C is the specimen retrieval device of FIG. 2A in an opened configuration.

FIG. 2D is the specimen retrieval device of FIG. 2A in a loaded configuration.

FIG. 2E is the specimen retrieval device of FIG. 2A in a compressed and elongated configuration.

FIG. 2F is the specimen retrieval device of FIG. 2A in an extracted configuration.

FIG. 3A is a specimen retrieval device in a storage configuration.

FIG. 3B is the specimen retrieval device of FIG. 3A in a deployed configuration.

FIG. 3C is the specimen retrieval device of FIG. 3A in a loaded configuration.

FIG. 3D is the specimen retrieval device of FIG. 3A in a compressed and elongated configuration.

FIG. 4A is a specimen retrieval device in a loaded configuration.

FIG. 4B is the specimen retrieval device of FIG. 4A in a compressed and elongated configuration.

FIG. 5 is a perspective view of a specimen retrieval device in a storage configuration.

FIG. 6A is a side view of the specimen retrieval device of FIG. 5 in a deployed configuration.

FIG. 6B is a partial perspective view of the specimen retrieval device of FIG. 5 in a deployed configuration.

FIG. 7A is a side view of the specimen retrieval device of FIG. 5 in an opened configuration.

FIG. 7B is a partial perspective view of the specimen retrieval device of FIG. 5 in an opened configuration.

FIG. 8 is a partial perspective view of the specimen retrieval device.

FIG. 9 is a perspective view of a portion of a handle including a string lock element.

FIG. 10A is a side view of the specimen retrieval device of FIG. 5 in a loaded configuration.

FIG. 10B is a partial perspective view of the specimen retrieval device of FIG. 5 in a loaded configuration.

FIG. 11A is a side view of the specimen retrieval device of FIG. 5 in a compressed and elongated configuration.

FIG. 11B is a partial perspective view of the specimen retrieval device of FIG. 5 in a compressed and elongated configuration.

FIG. 12 is a perspective view of a tightening mechanism.

FIG. 13 is a view of a specimen retrieval device.

FIG. 14 is a view of a portion of the specimen retrieval device of FIG. 13, illustrating an opening in a first containment member and an opening in a second containment member.

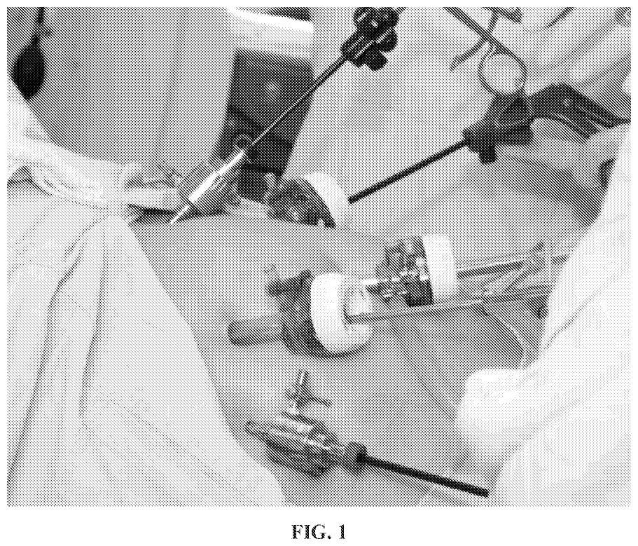

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that an apparatus comprises components A, B, and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and generally refer to humans. In some embodiments, the subject comprises a human who is undergoing a laparoscopic surgical procedure with a device as prescribed herein.

Unless otherwise defined, all technical terms used herein have the same meaning as commonly understood by one of ordinary skill in the art to which this disclosure belongs.

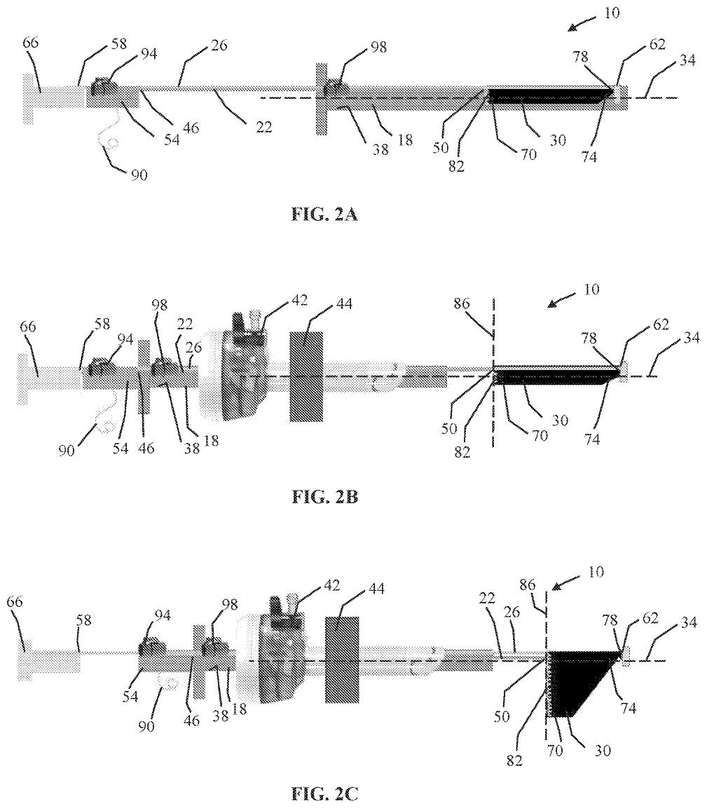

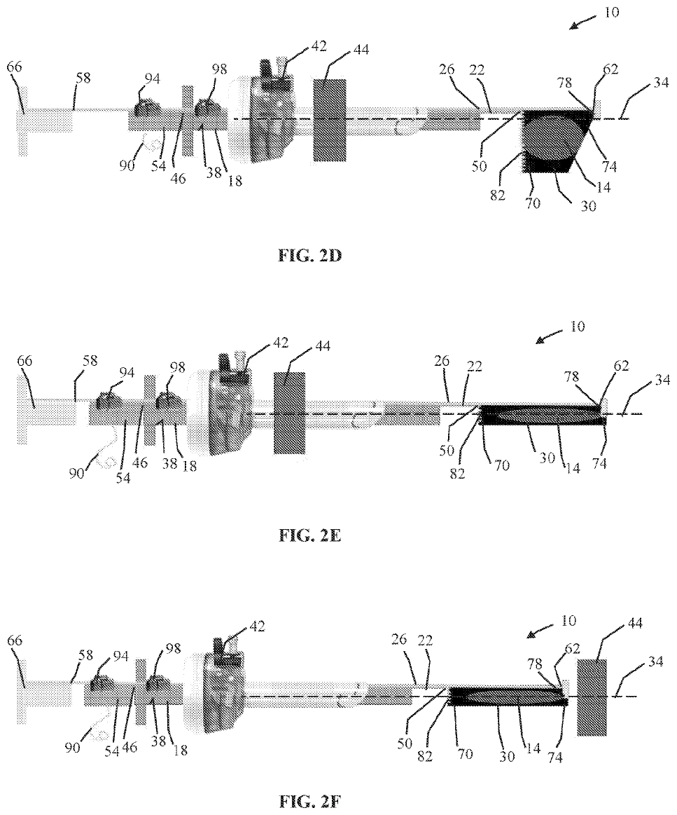

With reference to FIGS. 2A-2F, a specimen retrieval device 10 is a surgical tool used, for example, in a laparoscopic procedure to remove a specimen 14. The device 10 includes a sheath 18, a first rod 22, a second rod 26, and a specimen containment member 30.

With continued reference to FIG. 2A, the sheath 18 is a hollow tube that defines a longitudinal axis 34 and includes an internal passageway 38 that extends along the longitudinal axis 34. In some embodiments, the sheath 18 is made of medical grade plastic. In some embodiment, the sheath 18 has an outer diameter within a range of approximately 9 mm to approximately 10 mm and is configured to fit within an approximately 10 mm conventional trocar 42. In another embodiment, the sheath 18 has an outer diameter within a range of approximately 14 to approximately 15 mm and is configured to fit within an approximately 15 mm conventional trocar. Other dimensional ranges (e.g. 6-20 mm in diameter) are possible depending on the tissue type to be removed. In some embodiments, the sheath 18 includes an interior groove formed in a sidewall of the tube and at least one of the rods 22, 26 is received or nested within the interior groove. In some embodiments, a seal is formed within the sheath 18. The seal allows the rods 22, 26 to translate within the sheath 18 while allowing pressure to be maintained within the body cavity (pressure within the body cavity is an important component for visualization in laparoscopic surgery, for example). In some embodiments, the seal in the sheath 18 withstands pressures at least approximately 30 mm Hg. In some embodiments, the seal is positioned at a proximal end of the sheath, a distal end of the sheath, or at both ends. In some embodiments, the seal is one or more O-rings that form a sliding seal between the rods 22, 26 and the passageway 38 of the sheath 18.

With continued reference to FIG. 2A, the first rod 22 extends through the passageway 38 and includes a first proximal rod end 46 and a first distal rod end 50. In some embodiments, the first rod 22 is made of medical grade plastic. In other embodiments, the first rod 22 is made of a metal, such as a medical grade stainless steel. In the illustrated embodiment, a first rod handle 54 is positioned at the first proximal rod end 46. In some embodiments, the first rod handle 54 is ergonomic and made of medical grade plastic. The first rod handle 54 allows the user to manipulate the first rod 22 for movement along the longitudinal axis 34.

With continued reference to FIG. 2A, the second rod 26 extends through the passageway 38 and includes a second proximal rod end 58 and a second distal rod end 62. In some embodiments, the second rod 26 is made of medical grade plastic. In other embodiments, the second rod 26 is made of a metal, such as a medical grade stainless steel. In the illustrated embodiment, a second rod handle 66 is positioned at the second proximal rod end 58. In some embodiments, the second rod handle 66 is ergonomic and made of medical grade plastic. The second rod handle 66 allows the user to manipulate the second rod 26 for movement along the longitudinal axis 34. In some embodiments, the second rod handle 66 and the first rod handle 54 are identically shaped.

With continued reference to FIG. 2A, the specimen containment member 30 includes a proximal end 70 and a distal end 74 opposite the proximal end 70. In the illustrated embodiment, the proximal end 70 is positioned closer to the sheath 18 and the handles 54, 66 than the distal end 74 is. The proximal end 70 of the specimen containment member 30 is coupled to the first distal rod end 50 of the first rod 22. The distal end 74 of the specimen containment member 30 is coupled to the second distal rod end 62 of the second rod 26. In some embodiments, the distal end 74 of the specimen containment member 30 tapers to a distal fixation point 78 on the second rod 26.

As described in further detail herein, the second rod 26 is movable with respect to the first rod 22 to elongate the specimen containment member 30 along the longitudinal axis 34. Elongating (i.e., increasing the length) of the specimen containment member 30 along the axis 34 generates a compression on the specimen 14 contained therein. In the illustrated embodiment, the second distal rod end 62 moves away from the first distal rod end 50 to stretch and elongate the specimen containment member 30. In some embodiments, the device 10 includes a ratchet or other mechanically advantaged means of stretching apart the opposite ends 70, 74 of the specimen containment member 30.

In some embodiments, the specimen containment member 30 is flexible. In some embodiments, the specimen containment member 30 is impermeable (i.e., made of an impermeable material) to prevent fluid and/or material from penetrating through the walls of the specimen containment member 30. In some embodiments, the specimen containment member is a bag made of, for example, a medical grade thermoplastic or urethane. In some embodiments, the specimen containment member 30 includes a braided sleeve. The braided sleeve can aid in compression of a specimen 14 positioned within the specimen containment member 30 by translating linear motion created between the ends 70, 74 into radially compressive force to help reduce the diameter of the specimen 14. In some embodiments, the specimen containment member 30 achieves compressibility of the specimen 14 up to approximately 75%, depending on tissue type. In some embodiments, the braided sleeve is a weave. In some embodiments, the specimen containment member 30 is a braided sleeve with no secondary impermeable layer. In some embodiments, the braided sleeve is formed by rolling two ends of a braided tube back on top of themselves, similar to an Arthrex® finger trap, and fixing those two ends at or near the distal rod end 62. In some embodiments, the specimen containment member 30 is an elongated mesh bag. In some embodiments, the specimen containment member 30 includes a synthetic material (e.g., a synthetic mesh). In some embodiments, the specimen containment member 30 includes more than one layer of material. In some embodiments, the specimen containment member 30 is less than approximately 2 mm thick. In some embodiments, an entrance of the specimen containment member 30 flairs outward to help facilitate insertion of tissue into the specimen containment member 30.

With continued reference to FIG. 2A, the specimen containment member 30 includes an opening 82 positioned at the proximal end 70 of the specimen containment member 30. In some embodiments, a portion of the opening 82 is coupled to the first rod 22. In the illustrated embodiment, the second end 74 of the specimen containment member 30 is closed. In the illustrated embodiment, the opening 82 is oriented toward (e.g., facing toward) the sheath 18. In some embodiments, the opening 82 lies within a plane 86 that is perpendicular to the longitudinal axis 34. In some embodiments, a normal vector created by the opening 82 has a component that intersects plane 86. In other words, the opening 82 can open up at an angle that isn't exactly on the plane 86 but is partially directed toward the plane 86. In some embodiments, the longitudinal axis 34 intersects the opening 82. In some embodiments, the opening 82 is circular. In some embodiments, the second rod 26 is movable with respect to the first rod 22 to adjust a cross-sectional area of the opening 82. For example, in the illustrated embodiment, the second distal rod end 62 moves toward the first distal rod end 50 to increase the cross-sectional area of the opening 82. As such, when the second distal rod end 62 moves toward the first distal rod end 50, the specimen containment member 30 is longitudinally contracted or compacted (i.e., shortened along the longitudinal axis 34), causing the opening 82 to enlarge.

With continued reference to FIG. 2A, in some embodiments, a drawstring 90 is coupled to the opening 82 in the specimen containment member 30. The opening 82 is movable in response to actuation of the drawstring 90 between an open position (configured to permit a specimen 14 to pass through the opening 82) and a sealed position (configured to prevent a specimen 14 from passing through the opening 82). In other words, pulling on the drawstring 90 sinches closed the opening 82. As such, when the drawstring 90 is pulled tight, the opening 82 is reduced in size and is closed. In some embodiments, the drawstring 90 is a suture and/or string-like material. In some embodiments, the drawstring 90 is positioned as a loop through and around the opening 82 of the specimen containment member 30, forms a reversible knot (e.g., a slip), travels within the sheath 18 and then out of the first or second handle 54, 66. In other embodiments, the drawstring 90 extends out of a proximal end of the sheath 18. Where the user grasps the drawstring 90 includes, in some embodiments, a wafer/handle to aid in manipulation of the drawstring 90.

With continued reference to FIG. 2A, the device 10 includes a first lock 94. The first lock 94 is coupled between the first rod 22 and the second rod 26. The first lock 94 is movable between an unlocked position in which the first rod 22 is movable with respect to the second rod 26 and a locked position in which the first rod 22 is fixed relative to the second rod 26. The first lock 94 is a reversable locking mechanism used to prevent or allow translational motion between the rods 22, 26. In some embodiments, the first lock 94 includes a cam lever mechanism that can selectively lock relative positions of the first rod 22 and the second rod 26 in any configuration or relative arrangement. In other embodiments, the first lock 94 includes a rotational collet, a zip-tie, a ratchet mechanism, a latch, or other suitable locking mechanism.

With continued reference to FIG. 2A, the device 10 includes a second lock 98. The second lock 98 is coupled between the first rod 22 and the sheath 18. The second lock 98 is movable between an unlocked position in which the first rod 22 is movable with respect to the sheath 18 and a locked position in which the first rod 22 is fixed relative to the sheath 18. The second lock 98 is a reversable locking mechanism used to prevent or allow translational motion between the first rod 22 and the sheath 18. In some embodiments, the second lock 98 includes a cam lever mechanism that can selectively lock relative positions of the first rod 22 and the sheath 18 in any configuration or relative arrangement. In other embodiments, the second lock 98 includes a rotational collet, a zip-tie, a ratchet mechanism, a latch, or other suitable locking mechanism. In other embodiments, a locking pin and groove arrangement is provided between the first rod 22 (e.g., the first rod handle) and the sheath 18. In other embodiments, the second lock 98 selectively fixes either one or both of the first rod 22 and the second rod 26 relative to the sheath 18.

With reference to FIG. 2A, the device 10 is illustrated in a storage configuration with the specimen containment member 30 rolled or folded up into a storage shape and positioned within the sheath 18. In some embodiments, the dimensions of the specimen containment member 30 in the storage shape are approximately 8 mm by approximately 200 mm. Other storage configuration shapes are used depending on the tissue type to be removed.

With reference to FIG. 2B, the device 10 is illustrated in a deployed configuration with the device 10 inserted through a trocar 42 and into a body cavity through a body wall 44. To deploy, an axial force is applied to the handles 54, 66 and rods 22, 26. In some embodiments, the second lock 98 is moved to a locked position after deployment to fix the first rod 22 relative to the sheath 18.

With reference to FIG. 2C, the device 10 is illustrated in an opened configuration with the second rod 26 is pulled backwards (i.e., to the left in the frame of view of FIG. 2C; e.g., rod end 62 moves toward the abdominal wall and handle 66 moves away from the abdominal wall) with an axial force by the user. In other words, the second rod 26 moves with respect to the first rod 22 to adjust the overall size and shape of the opening 82 and the specimen containment member 30. In the open configuration, the specimen containment member 30 is shortened, and the cross-sectional area of the opening 82 increases such that a specimen 14 can be placed within the internal volume of the specimen containment member 30. In the opened configuration, the opening 82 of the specimen containment member 30 is enlarged and the drawstring 90 is opened as well, with a length of the drawstring being taken up in the enlarged opening 82 circumference. In some embodiments, the dimensions of the specimen containment member 30 in the opened configuration are approximately 40-60 mm by approximately 75-150 mm. In some embodiments, the opening 82 of the specimen containment member 30 in the opened configuration is within a range of approximately 40 mm to approximately 60 mm in diameter. During use, both or either of the first lock 94 and the second lock 98 can be moved to a locked position to maintain an optimal orientation, shape, and/or position of the specimen containment member 30 within the body cavity. In the opened configuration, the first handle 54 is spaced from the second handle 66.

With reference to FIG. 2D, the device 10 is illustrated in a loaded configuration with a specimen 14 positioned within the specimen containment member 30. The specimen 14 is inserted by a surgeon into the specimen containment member 30 through the enlarged opening 82. In some embodiments, a laparoscopic tool or instrument is used to assist with positioning the specimen 14 within the specimen containment member 30. In some embodiments, the first lock 94 is in the locked position while the specimen 14 is positioned within the specimen containment member 30 such that the first rod 22 is fixed relative to the second rod 26. In some embodiments, the first lock 94 and the second lock 98 are in the locked position while the specimen 14 is positioned or loaded within the specimen containment member 30.

With reference to FIG. 2E, the device 10 is illustrated in a compressed and elongated configuration with the specimen 14 elongated along the longitudinal axis 34 and compressed. After the specimen 14 is loaded into the specimen containment member 30, the drawstring 90 is pulled to cinch close the opening 82. Then, with the first lock 94 in the unlocked position, the user applies an axial depressing force to the second rod 26 (i.e., to the right in the view of FIG. 2E) to cause radial compression of the specimen containment member 30. In some embodiments, the user applies a pulling axial force to the first rod 22 (i.e., to the left in the view of FIG. 2E) to cause elongation and radial compression of the specimen containment member 30. During compression of the specimen 14, the second handle 66 is moved closer to the first handle 54. After the desired compression of the specimen 14 is achieved, the locks 94, 98 are moved to a locked position to allow the user to be relieved of persistent manual compression via the axial loading. Advantageously, if the user in working in a small space, the specimen containment member 30 can be partially elongated and then moved to an area with more room before being compressed further. As such, the device 10 provides compresses the specimen 14 while enabling an insufflated abdominal or other body cavity.

With reference to FIG. 2F, the device 10 is illustrated in an extracted configuration with the device 10 removed from the body cavity through the body wall 44. In some embodiments, the compressed specimen 14 is removed through the trocar 42. In other embodiments, the compressed specimen 14 is removed with the trocar 42 at the same time. In some embodiments, the trocar 42 is re-used or continually used after the extraction of the specimen 14 and the device 10. In some embodiments, the specimen containment member 30 is removable from the rods 22, 26.

In the illustrated embodiment, the device 10 is a single use specimen retrieval tool configured to reduce the cross-sectional area and maintain alignment of the specimen 14 along an axis of removal (i.e., the longitudinal axis 34), thereby increasing ease of extraction. In some embodiments, the device 10 is prepackaged, sterilized, single use. The opening 82 is at the proximal end 70 of the specimen containment member 30 and is oriented toward the sheath 18 such that fluid from the specimen 14 during compression and extraction does not drip into the body cavity. In other words, the proximal opening 82 advantageously keeps specimen fluid within the specimen containment member 30. In some embodiments, the opening 82 is oriented towards the laparoscopic port site such that the entrance of the opening 82 is the first portion of the specimen containment member that is removed from the patient during tissue extraction, which reduces the chances of specimen contamination within the body of the patient. In some embodiments, the opening 82 is oriented toward the laparoscopic port site to aid with loading the specimen. A specimen opening facing away from the laparoscopic port site may be difficult to load during laparoscopic surgery due to the difficulties in visualization and working against gravity while loading the specimen.

A method of removing a specimen from a body cavity is disclosed herein with FIGS. 2A-2F illustrating the various steps of the method and the corresponding configuration of the device. In some embodiments, the method includes storing the device 10 (FIG. 2A), deploying the device 10 in a body cavity (FIG. 2B), opening the opening 82 in the specimen containment member 30 (FIG. 2C), loading the specimen containment member 30 with the specimen 14 (FIG. 2D), compressing and elongating the specimen containment member 30 and the specimen 14 (FIG. 2E), and extracting the specimen 14 and device 10 from the body cavity (FIG. 2F).

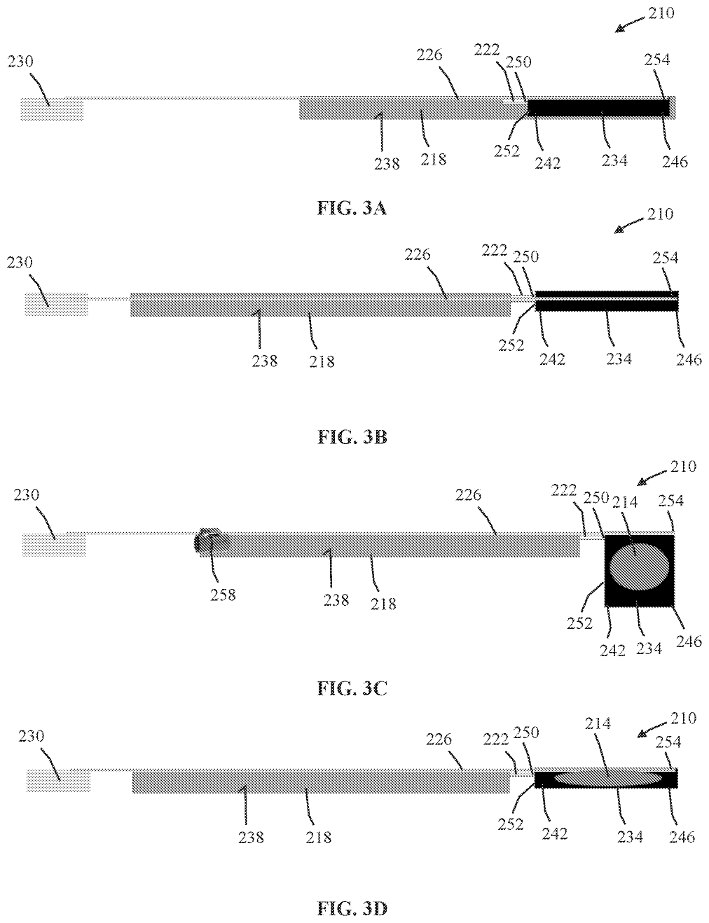

With reference to FIGS. 3A-3D, a specimen retrieval device 210 is a surgical tool used, for example, in a laparoscopic procedure to remove a specimen 214. The device 210 includes a sheath 218, a first rod 222, a second rod 226, a handle 230, and a specimen containment member 234. The sheath 218 includes a passageway 238 and the second rod 226 extends through the passageway 238.

With reference to FIG. 3A, the specimen containment member 30 includes a proximal end 242 and a distal end 246 opposite the proximal end 242. In the illustrated embodiment, the proximal end 242 is closer to the sheath 218 and the handle 230 than the distal end 246. The proximal end 242 of the specimen containment member 234 is coupled to a first distal rod end 250 of the first rod 222 and includes an opening 252. The distal end 246 of the specimen containment member 234 is coupled to a second distal rod end 254 of the second rod 226. As described herein, the second rod 226 is movable with respect to the sheath 218 and the first rod 222 to elongate the specimen containment member 234. In the illustrated embodiment, the second distal rod end 254 moves away from the sheath 218 to stretch and elongate the specimen containment member 234.

With reference to FIG. 3A, the device 210 is illustrated in a storage configuration with the specimen containment member 234 positioned within the sheath 218. The first rod 222 is positioned within the passageway 238 when the device 210 is in the storage configuration.

With reference to FIG. 3B, the device 210 is illustrated in a deployed configuration with the specimen containment member 234 and the first rod 222 positioned outside of the sheath 218. In the illustrated embodiment, the first rod 222 slides relative to the sheath 218 and locks the proximal end 242 of the specimen containment member 234 relative to the sheath 218. In some embodiments, this is accomplished with an annular snap fit that is integrated into the sheath 218. In some embodiments, the first rod 222 offsets the opening 252 from the sheath 218 to make it easier to load the specimen 214 into the specimen containment member 234.

With reference to FIG. 3C, the device 210 is illustrated in a loaded configuration with the specimen 214 positioned within the specimen containment member 234. The second rod 226 is pulled back to expand the opening 252 for easier loading of the specimen 214. In some embodiments, the device 210 includes a lock 258 (e.g., a collet, clamp, lever, ratchet, etc.) to allow the second rod 226 to be held in place relative to the sheath 218.

With reference to FIG. 3D, the device 210 is illustrated in a compressed and elongated configuration with the specimen 234 elongated and compressed. The second rod 226 is pushed forward (i.e., to the right in the view of FIG. 3D) to stretch and compress the specimen 214 in the specimen containment member 234. In some embodiments, the lock 258 is then placed in a locked position to help maintain the compression on the specimen 214. After elongating the specimen containment member 234 and compressing the specimen 214, the device 210 and the compressed specimen 214 are removable from a small laparoscopic incision.

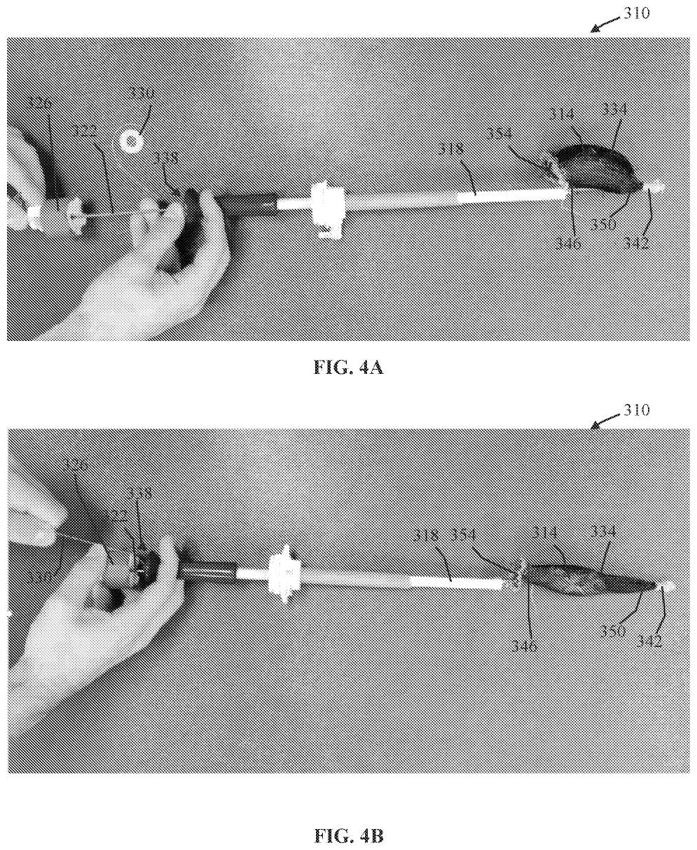

With reference to FIGS. 4A-4B, a specimen retrieval device 310 is a surgical tool used, for example, in a laparoscopic procedure to remove a specimen 314. The device 310 includes a sheath 318, a rod 322, a handle 326, a drawstring 330, and a specimen containment member 334.

The sheath 318 includes a passageway 338 and the rod 322 extends through the passageway 338. The rod 322 includes a distal rod end 342 coupled to the specimen containment member 334. In some embodiments, the distal rod end 342 is an atraumatic tip. The specimen containment member 334 includes a proximal end 346 and a distal end 350 opposite the proximal end 346. In the illustrated embodiment, the distal end 350 is closed and is coupled to the distal rod end 342. The proximal end 346 of the specimen containment member 334 includes an opening 354 and is coupled to the sheath 318. In other words, the opening 354 positioned at the proximal end 346 of the specimen containment member 334. The opening 354 is oriented toward the sheath 318. As described herein, the rod 322 is movable with respect to the sheath 318 to compress the specimen containment member 334. In the illustrated embodiment, the distal rod end 342 moves away from the sheath 318 to elongate the specimen containment member 334 and to compress and align the specimen 314. In some embodiments, the specimen containment member 334 is flexible, impermeable, and/or includes a braided sleeve.

With reference to FIG. 4A, the device 310 is illustrated in a loaded configuration with the specimen 314 positioned within the specimen containment member 334. Similar to the other devices 10, 210 described herein, the rod 322 is movable with respect to the sheath 318 to adjust a cross-sectional area of the opening 354. The distal rod end 342 moves toward the sheath 318 to increase the cross-sectional area of the opening 354.

With reference to FIG. 4B, the device 310 is illustrated in a compressed configuration with the specimen 314 stretched and compressed. The rod 322 is moved with respect to the sheath 318 to compress the specimen 314. In the illustrated embodiment, the distal rod end 342 moves away from the sheath 318. The drawstring 330 is pulled to cinch close the opening 354.

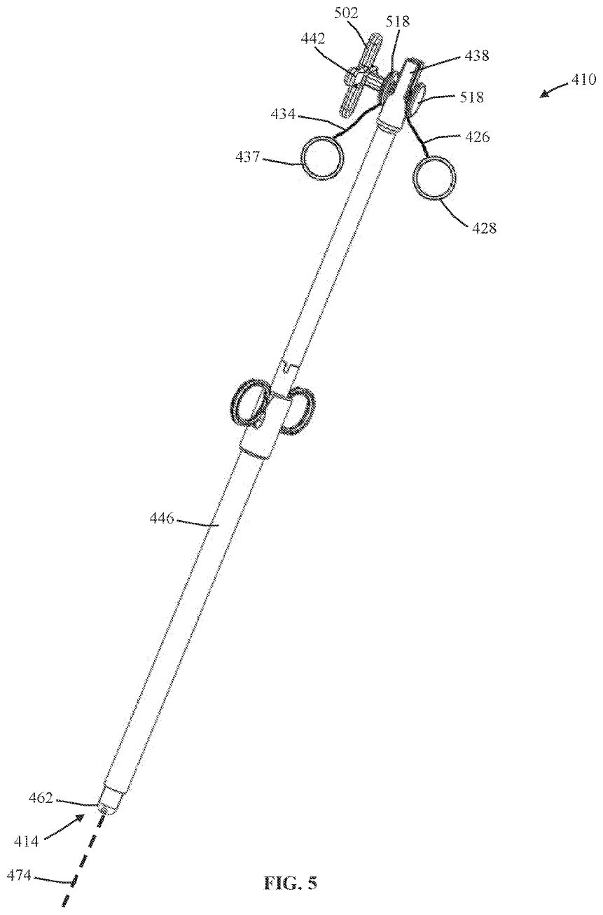

With reference to FIGS. 5-12, a specimen retrieval device 410 is a surgical tool used, for example, in a laparoscopic procedure to remove a specimen 412. The device 410 includes a rod assembly 414, a first containment member 418 (e.g., a compression mesh), a second containment member 422 (e.g., a containment bag), a plurality of expansion strings 426, a compression string 430, a drawstring 434, a handle 438, and a tightening mechanism 442. In some embodiments, the device 410 includes a sheath 446 defining a passageway 450. In the illustrated embodiment, the rod assembly 414 is positioned at least partially within the passageway 450 and the strings 426, 430, 434 extend through the passageway 450.

With reference to FIG. 5, the device 410 is illustrated in a storage configuration with the containment members 418, 422 positioned within the sheath 446. With the device inserted into the patient, for example inserted into the abdominal cavity, the user can advance the rod assembly 414 distally through the outer sheath 446 until the containment members 418, 422 are exposed. In the illustrated embodiment, the rod assembly 414 is positioned at least partially within the passageway 450 of the sheath 446 when the device 410 is in the storage configuration. In some embodiments, the outer sheath 446 is made of a medical grade thermoplastic or metal. In some embodiments, the outer sheath 446 has a diameter within a range of approximately 8 mm to approximately 15 mm and a length within a range of approximately 150 to approximately 350 mm. In other embodiment, the outer sheath 446 is sized in accordance with the tissue specimen to be collected.

In the illustrated embodiment, the rod assembly 414 (also referred to herein as “a rod”) includes a support rod 454, an elongated body 458 coupled to the support rod 454, and a tip 462 at the distal end of the support rod 454. In the illustrated embodiment, the tip 462 is an atraumatic tip. The rod assembly 414 includes a channel 466 (e.g., groove, passageway, etc.) formed in the tip 462. In other embodiments, the channel 466 is any means of allowing the strings 426 to change directions. In some embodiments, the rod assembly 414 includes a rod passageway 470 and the strings 426, 430, 434 extend through the rod passageway 470. In some embodiments, the rod passageway 470 is formed in the elongated body 458. In some embodiments, the elongated body 458 has a larger diameter than a support rod 454. The rod assembly 414 defines a longitudinal axis 474 and the rod passageway 470 extends along the longitudinal axis 474. In some embodiments, the elongated body 458 is made of a medical grade thermoplastic and is of a diameter smaller than the outer sheath 446 such that the elongated body 458 slides within the outer sheath 446. In some embodiments, the elongated support rod 454 is made of a medical grade metal such as stainless steel.

With continued reference to FIG. 5, the device 410 may initially be in a stored configuration where the first and second containment members 418, 422 are contained (or at least partially contained) within the outer sheath 446. In some configuration the outer sheath is omitted. In the illustrated embodiment, the device 410 is endoscopically inserted into the patient. In particular, the atraumatic tip 462 is inserted first followed by the outer sheath 446 and the other components housed within the outer sheath 446. In some embodiments, the device 410 is configured to be inserted through a trocar or through an opening in the patient.

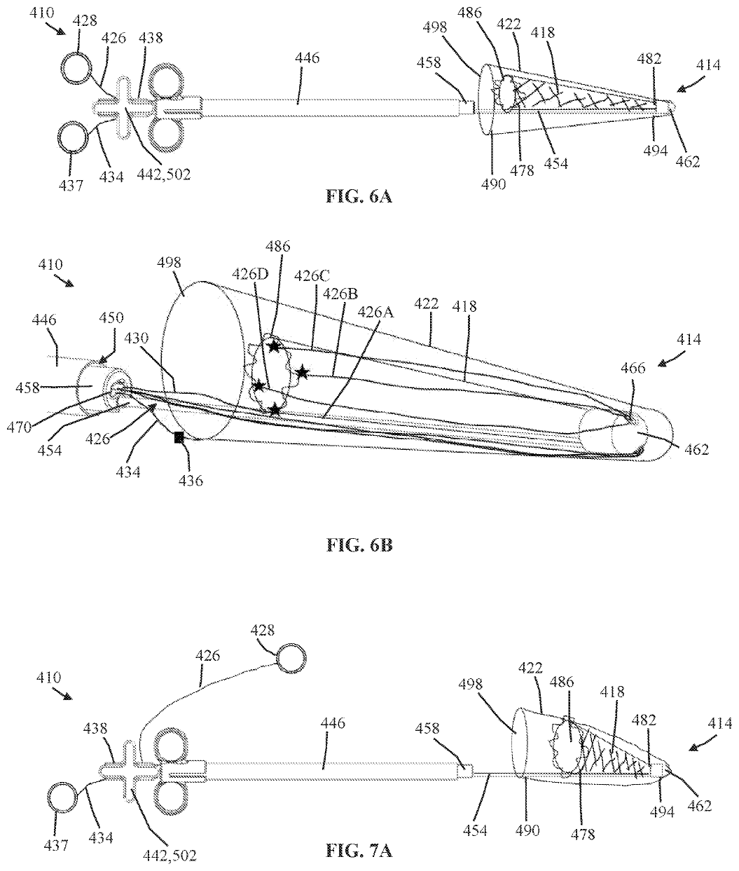

With reference to FIGS. 6A and 6B, the device 410 is illustrated in a deployed configuration with the containment members 418, 422 positioned outside of the sheath 446.

In the illustrated embodiment, the first containment member 418 includes a first end 478 and a second end 482 opposite the first end 478. The second end 482 is coupled to the tip 462 and an opening 486 is positioned at the first end 478. In the illustrated embodiment, the opening 486 in the containment member 418 is oriented toward the sheath 446 and the handle 438 (e.g., proximally oriented). In some embodiments, the opening 486 faces or lies within a plane that is perpendicular to the longitudinal axis 474 of the rod assembly 414.

In some embodiments, the containment member 418 is a compression mesh sleeve. In some embodiments, the mesh sleeve is braided, knit, or woven. In some embodiments, the mesh is a medical grade thermoplastic. Multiple configurations of mesh may be utilized depending on the type of tissue to be extracted from the patient. For example, a mesh made from a plain weave oriented at approximately 45 degrees composed of monofilament fibers with a diameter within a range of approximately 0.1 mm and approximately 0.4 mm. The monofilament fibers are doubled over on themselves and compose an overall mesh sleeve with a longitudinal length within a range of approximately 80 mm to approximately 200 mm. In other embodiments, the containment member 418 is a bag made of flexible medical grade materials such as a thermoplastic or urethane. Other configurations may be useful depending on the specific tissue type to be removed. In the illustrated embodiment, second end 482 (e.g., the distal end) of the containment member 418 is fixed at the atraumatic tip 462 with, for example, medical grade adhesive. The first end 478 (e.g., the proximal end) of the containment member 418 selectively translates about, along, or adjacent to the elongated support rod 454.

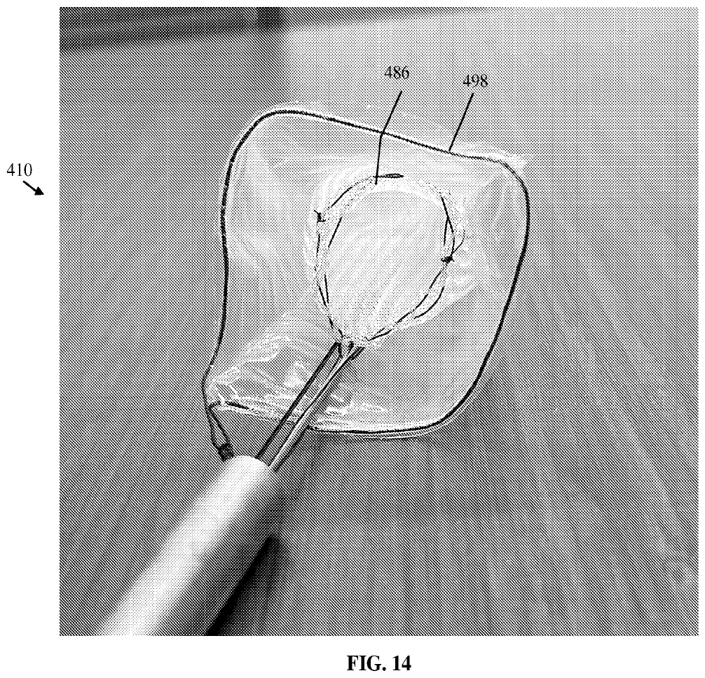

In the illustrated embodiment, the first containment member 418 is positioned at least partially within the second containment member 422. The second containment member 422 includes a third end 490 (e.g., a proximal end) and a fourth end 494 (e.g., a distal end) opposite the third end 490. The second containment member 422 includes a second opening 498 positioned at the third end 490. The second opening 498 is generally oriented toward the proximal end of the device 410. In some embodiments, the second opening 498 has a diameter within a range of approximately 40 mm and approximately 80 mm. In some embodiments, the second containment member 422 is impermeable to fluid, such that fluid from the specimen 412 in the first containment member 418 does not leak into the body cavity during removal. In some embodiments, the second containment member 422 is a flexible bag made of a medical grade plastic or urethane. In the illustrated embodiment, the second containment member 422 is translucent or transparent so that a user can visualize the configuration of the enclosed first containment member 418. In the illustrated embodiment, the second containment member 422 completely contains the first containment member 418 to minimize the leaking of fluids during subsequent tissue specimen removal. With reference to FIG. 14, the device 410 is illustrated with the opening 486 in the first containment member 418 contained within the opening 498 in the second containment member 422. In other embodiments, there is only a single containment member (e.g., no second containment member).

As detailed herein, the drawstring 434 is coupled to the second opening 498 and the second opening 498 at least partially closes in response to actuation of the drawstring 434. In some embodiments, the drawstring 434 is a medical grade braided suture, such as nylon. In some embodiments, the drawstring 434 includes a slip knot 436 positioned near the second containment member 422. In the illustrated embodiment, the drawstring 434 includes a graspable member 437 coupled to an end of the drawstring 434.

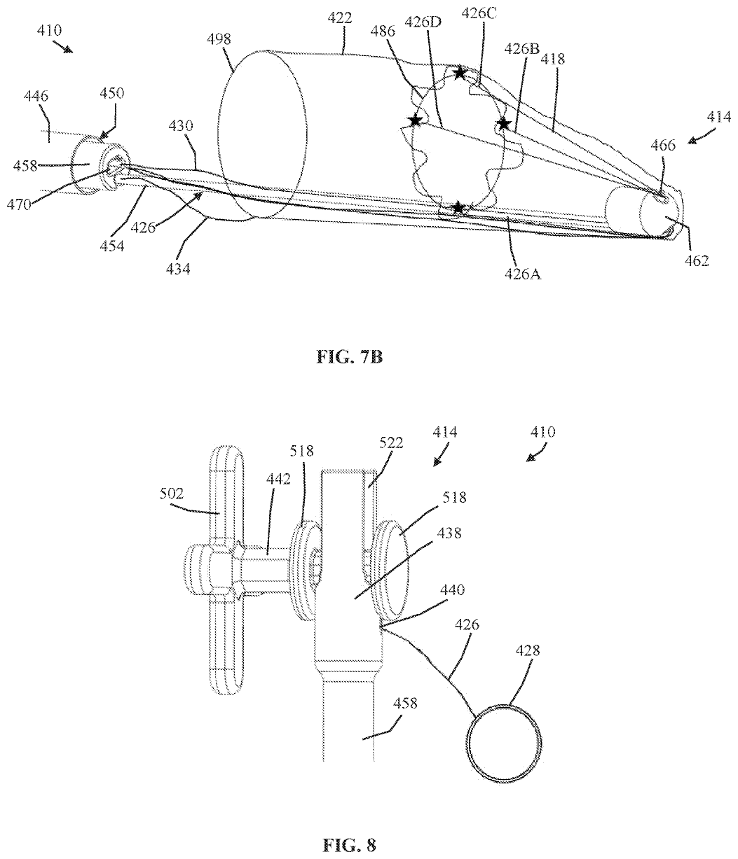

With continued reference to FIG. 6B, the device 410 includes the plurality of strings 426 (e.g., mesh expansion strings) coupled to the opening 486 of the containment member 418. In the illustrated embodiment, the plurality of strings 426 extend through the channel 466 formed in the tip 462. In other words, the strings 426 extend distally from the opening 486 toward the tip 462 and then back proximally toward the handle 438. The strings 426 extending a first direction along the longitudinal axis 474 and a second opposite direction along the longitudinal axis 474. In other words, the expansion strings 426 are routed from their connection points at the first opening 486 distally towards the atraumatic tip 466, routed through the tip channel 466, and then then directed back towards the proximal end of the device 410 through the rod passageway 470. As detailed further herein, the opening 486 enlarges in response to actuation of the plurality of strings 426 (FIG. 7B), which advantageously make it easier to position the specimen 412 within the containment member 418.

With continued reference to FIG. 6B, in the illustrated embodiment the plurality of strings 426 includes a first string 426A, a second string 426B, a third string 426C, and a fourth string 426D. The strings 426 are spaced apart from one another along the circumference of the opening 486. In other words, the connection points between the strings 426 and the opening 486 are distributed about the circumference of the compression mesh opening 486. Depending on the quantity and radial orientation of the string connection points, the compression mesh can be made to expand or angle in certain directions amenable to the procedure. In some embodiments, the second string 426B is coupled to the opening 846 within a range of approximately 90 degrees to approximately 120 degrees from the first string 426A. In some embodiments, the third string 426C is coupled to the opening 486 within a range of approximately 60 degrees to approximately 90 degrees from the second string 426B. In some embodiments, the fourth string 426D is coupled to the opening 486 within a range of approximately 60 degrees to approximately 90 degrees from the third string 426C.

In the illustrated embodiment, the first string 426A and second string 426B are positioned approximately 120 degrees apart; the second string 426B and the third string 426C are positioned approximately 60 degrees apart; and the fourth string 426D and the third string 426C are positioned approximately 60 degrees apart. In other words, there are three compression mesh expansion strings 426B, 426C, 426D located at approximately 120, 180, and 240 degrees from the elongated support rod 454 and a fourth compression mesh expansion string 426A located at approximately 0 degrees from the elongated support rod 454. In some embodiments, the opening 486 flexes or re-orients, in addition to enlarging, at an angle away from the support rod 454 in response to actuation of the strings 426. In other words, the strings 426 are secured around the circumference of the opening 486 such that actuation of the strings 426 causes the opening 486 to enlarge and angle away from the longitudinal axis 474 of the rod assembly 414. Angling the opening 486 in response to actuation of the strings 426 advantageously makes it easier to position the specimen 412 within the containment member 418. In other words, the illustrated configuration biases the first containment member 418 to open partially out and away from the elongated support rod 454, facilitating more facile placement of the tissue specimen 412 into the first containment member 418.

With reference to FIGS. 7A and 7B, the device 410 is illustrated in an opened configuration. With the device 410 in place within the body of the patient and the tissue specimen 412 excised, the user can next pull on the compression mesh expansion strings 426. In the opened configuration, the plurality of expansion strings 426 is actuated and the opening 486 of the first containment member 418 is enlarged. In the illustrated embodiment, the plurality of strings 426 is coupled together with a graspable member 428 coupled to an end of the strings 426.

To open or expand the first containment member 418, the expansion strings 426 are actuated by a user. As a result, the first containment member 418 pulls distally when tension is placed on the expansion strings 426. As the first containment member 418 is contracted (e.g., the relative distance between the proximal end and distal end of the first containment member 418 is reduced) the orientation of the woven fibers in the compression mesh become more perpendicular to the longitudinal axis 474 and the first containment member 418 expands in diameter. As such, loading of the tissue specimen 412 is facilitated while reducing the overall size of the first containment member 418, which can further aid in the insertion and removal through the laparoscopic port site.

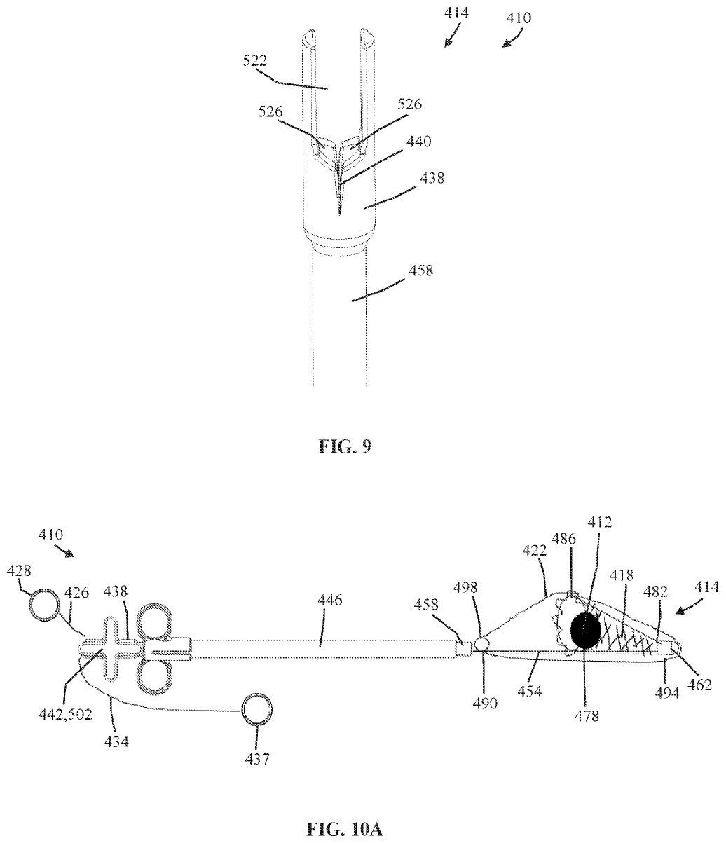

With reference to FIGS. 8 and 9, the handle 438 is coupled to a proximal end of the rod assembly 414. The handle 438 includes a string lock element 440 (e.g., a slot) formed therein that is configured to receive at least one string. In the illustrated embodiment, the slot 440 is configured to releasably secure the plurality of strings 426. After actuating the plurality of strings 426 to enlarge the opening 486, a user may position one or more of the strings 426 in the slot 440 to temporarily keep the opening 486 enlarged even after releasing user-applied force on the plurality of strings 426. In other words, when the expansion strings 426 are placed under tension to expand open the first containment member 418, the user can then slide those string(s) into the string lock element 440 to maintain the tension on the string(s). This allows the first containment member 418 to remain in the tissue receiving configuration while the user can now release the strings 426 and use a free hand to load the tissue specimen 412 into the first containment member 418. In the illustrated embodiment, the string lock element 440 consists of a wedged slot (e.g., channel) that creates an interference fit between the slot and the string when the string is positioned in the slot. In some embodiments, the string lock element 440 is released by pulling the string out of the slot. In other embodiments, the string lock element 440 includes wrapping the string around a post, tying the string, sliding the string through a gromet, pulling the string through an elastomeric seal to increase friction, and other suitable means.

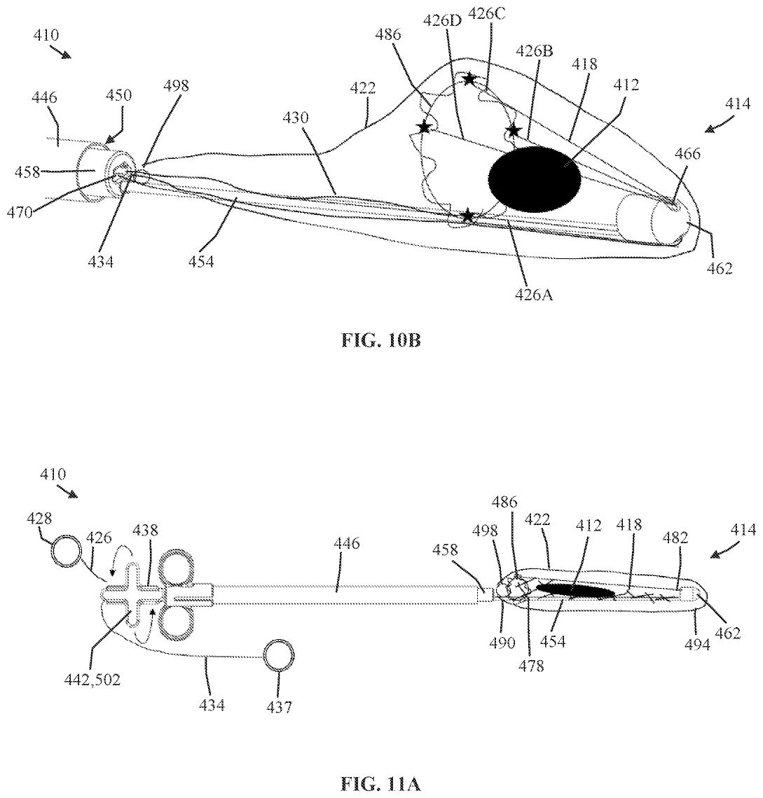

With reference to FIGS. 10A and 10B, the device 410 is illustrated in a loaded configuration with the specimen 412 positioned within the specimen containment member 418 and the outer containment member 422. When the tissue specimen 412 is loaded inside the first containment member 418 and the second containment member 422, the drawstring 434 is tensioned by a user. The tension in drawstring 434 causes the opening 498 of the second containment member 422 to close, or at least partially close, as the slip knot 436 is pulled tighter. The second containment member 422 advantageously contains any fluid associated with the tissue specimen 412 and provides a low friction surface around the compression mesh which can help facilitate subsequent removal of the specimen from the patient.

In some embodiments, once the specimen 412 is placed inside the first containment member 418, the user may (but is not required to) release the tension from the mesh expansion strings 424 and let the first containment member 418 relax to an unstressed state. In the illustrated embodiment, with the expansion strings 426 releasably secured in the friction slot 440, if the user forgets to release the mesh expansion strings 426, the strings 426 will tend to release on their own automatically due to opposing pull directions.

With reference to FIGS. 11A and 11B, the device 410 is illustrated in a compressed and elongated configuration with the specimen 412 elongated and compressed. In the illustrated embodiment, the compression string 430 is looped around (e.g., routed about) the circumferential opening 486 of the first containment member 418, through the rod passageway 470 and connected to the user-actuated tightening mechanism 442. With the containment bag drawstring 434 tightened and the second opening 498 closed or partially closed, the user can next actuate the compression string 430 to elongate the first containment member and compress the specimen 412 enclosed therein. In the illustrated embodiment, the compression string 430 is coupled to the user-actuated tightening mechanism 442. As detailed herein, the specimen 412 positioned within the first containment member 418 is compressed in response to actuation of the compression string 430 by the tightening mechanism 442.

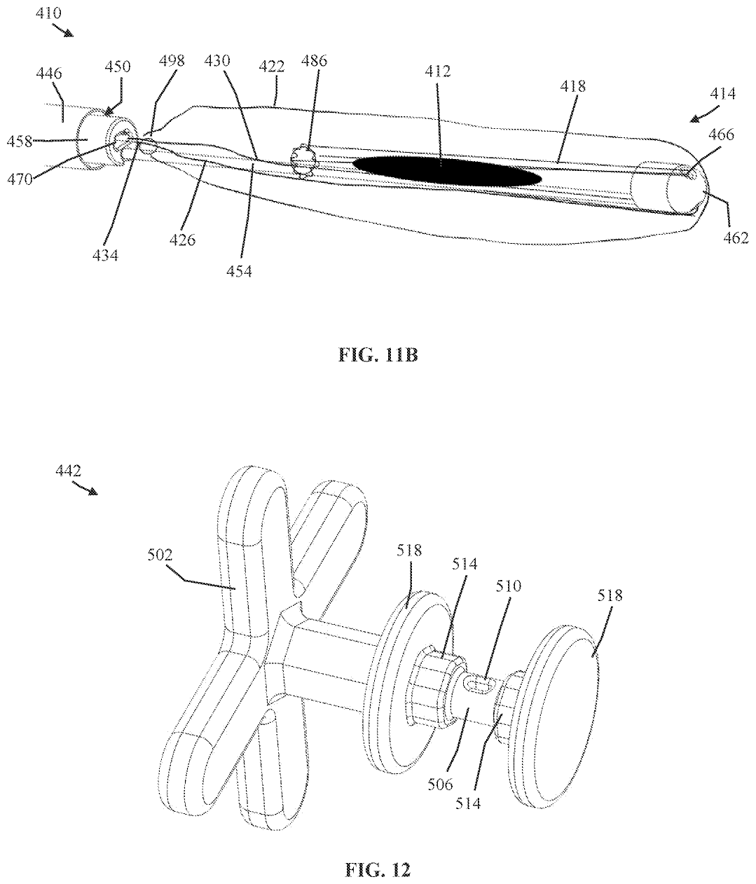

With reference to FIGS. 8 and 12, the tightening mechanism 442 includes a knob 502 (e.g., a handle), a post 506, a string mount 510 on the post 506, a faceted circumferential surface 514, and channel alignment members 518. In some embodiments, the compression string 430 is pre-fixed to the string mount 510.

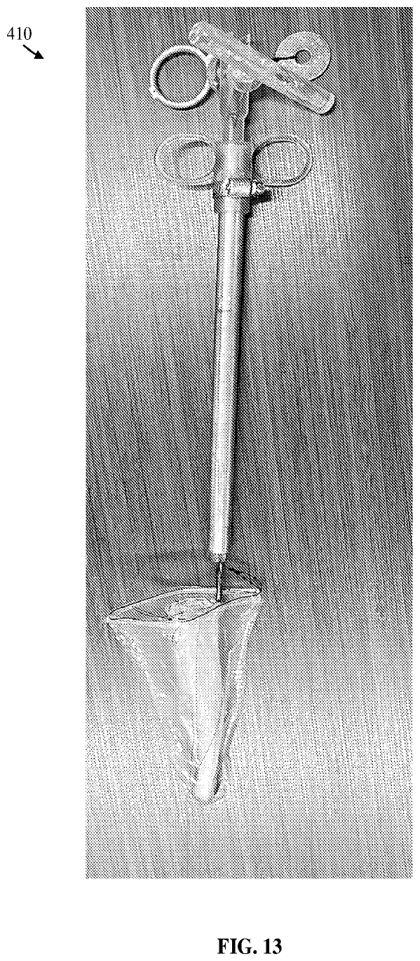

When the user wants to compress down the tissue specimen 412 within the first containment member 418, the user actuates the tightening mechanism 442. In the illustrated embodiment, the user rotates the knob 502 and as the knob 502 rotates, the compression string 430 is wound around the post 506. In the illustrated embodiment, the knob 502 has four extensions. With reference to FIG. 13, the device 410 is shown with a knob having two extensions.

As the compression string 430 wraps around the tightening post 506, the string 430 pulls on the containment member 418 is a proximal direction. As the compression string 430 is actuated, the first opening 486 of the first containment member 418 is closed or at least partially closed because the compression string 430 is looped or routed about the circumference of the first opening 486. In other words, the first opening 486 at least partially closes in response to actuation of the compression string 430. Simultaneously, the first containment member 418 is elongated and the fibers of the mesh weave align along the longitudinal axis 474. This reduces the overall diameter of the compression mesh 418, creating a circumferential compressive force around the tissue specimen 412. In other words, the specimen 412 positioned within the first containment member 418 is compressed in response to actuation of the compression string 430 by the user activating the tightening mechanism 442.

With reference to FIG. 9, the handle 438 further includes a groove 522 that at least partially receives the user-actuated tightening mechanism 442. In some embodiments, the handle 438 is formed as part of the elongated body 458 and is part of the rod assembly 414. In operation, the faceted circumferential surface 514 is rotatable relative to the groove 522. Specifically, the faceted circumferential surface 514 abuts engagement surfaces 526 formed in the end of the groove 522 to selectively secure the tightening mechanism 442 in position. As tension is applied to the compression string 430, the faceted circumferential surface 514 is pulled against the engagement surfaces 526 in the groove 522 on the handle 438. In other words, the planar facets of the circumferential surface 514 abut the engagement surfaces 526 to prevent or halt rotation of the post 506. This increases the force required to change the position of the knob 502. As such, the groove 522 and the faceted circumferential surface 514 advantageously allow the user to adjust the amount of tension placed on the first containment member 418 without having to hold onto the knob 502 to maintain tension. The tightening mechanism 442 also provides mechanical advantage to the tightening of the first containment member 418, allowing the user to further compress difficult to remove tissues by rotation of the knob 502. The channel alignment members 518 on the tightening mechanism 442 are positioned on opposite sides of the groove 522 and keep the tightening mechanism 442 aligned with respect to the elongated body 458.

In some embodiments, the elongated support rod 454 provides a visual cue to the user when maximum tension is reached by the tightening mechanism 442 by starting to deflect. Depending on the desired tissue compression, the stiffness and dimensions of the elongated support rod 454 can be selected to provide the visual bending cue at the proper tension range.

Other mesh compression tightening mechanisms are contemplated, including but not limited to an integrated rotational ratcheting system, such as the BOA dial system by BOA Technology Inc., other ratcheting systems, a pulley system routed to provide mechanical advantage, and others. In some embodiment, no mechanical advantage is provided and the user pulls directly on the compression mesh tightening string and selectively locks it in place using, for example, a string lock element.

After elongating the first containment member 418 and compressing the specimen 412, the device 410 and the compressed specimen 412 are removable from a small laparoscopic incision. In other words, once the tissue specimen 412 is compressed, the user can then easily remove the device 410 and the tissue specimen 412 from the patient body cavity.

As such, the devices 10, 210, 310, 410 disclosed herein are used to remove a specimen (e.g., a gallbladder) during minimally invasive surgery (e.g., laparoscopic, robotic, VATC, retroperitonescopy) without the need to enlarge incisions on the patient or risk the rupture of the specimen bag due to difficult extraction.

One skilled in the art will readily appreciate that the present disclosure is well adapted to carry out the objects and obtain the ends and advantages mentioned, as well as those inherent herein. The present disclosure described herein are exemplary embodiments and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Various features and advantages are set forth in the following claims.

What is claimed is:

1. A device comprising:
a rod with a tip and a channel formed in the tip;
a containment member including a first end and a second end opposite the first end; wherein the second end is coupled to the tip; and wherein the containment member includes an opening positioned at the first end; and
a string coupled to the opening of the containment member, the string extends through the channel in the tip.

2. The device of claim 1, further including a sheath defining a passageway; wherein the rod is positioned at least partially within the passageway and movable with respect to the sheath; and wherein the string extends through the passageway; wherein the opening in the containment member is oriented toward the sheath.

3. The device of claim 1, wherein the rod includes a rod passageway, and wherein the string extends through the rod passageway, and wherein the string includes a graspable member coupled to an end of the string.

4. The device of claim 1, wherein the containment member is a first containment member and the device further includes a second containment member; wherein the first containment member is positioned at least partially within the second containment member.

5. The device of claim 4, wherein the first containment member is a mesh sleeve.

6. The device of claim 5, wherein the second containment member is impermeable to fluid.

7. The device of claim 4, wherein the second containment member includes a third end and a fourth end opposite the third end, wherein the second containment member includes a second opening positioned at the third end.

8. The device of claim 7, wherein a drawstring is coupled to the second opening; wherein the second opening at least partially closes in response to actuation of the drawstring.

9. The device of claim 1, wherein the string is a first string and the device further includes a second string coupled to the opening of the containment member; wherein the second string is further coupled to a user-actuated tightening mechanism.

10. The device of claim 9, wherein the opening of the containment member enlarges in response to actuation of the first string, and wherein a specimen positioned within the containment member is compressed in response to actuation of the second string.

11. The device of claim 1, wherein the string is one of a plurality strings coupled to the opening of the containment member, wherein the opening enlarges in response to actuation of the plurality of strings.

12. The device of claim 1, wherein the string is a first string, and the device further includes a second string, a third string, and a fourth string; wherein the second string is coupled to the opening within a range of 90 degrees to 120 degrees from the first string, the third string is coupled to the opening within a range of 60 degrees to 90 degrees from the second string, and the fourth string is coupled to the opening within a range of 60 degrees to 90 degrees from the third string.

13. The device of claim 1, wherein the rod defines a longitudinal axis and the opening faces a plane that is perpendicular to the longitudinal axis.

14. The device of claim 1, further comprising a handle coupled to the rod, wherein the handle includes a slot configured to releasably secure the string.

15. A device comprising:
a rod with a tip;
a containment member including a first end and a second end opposite the first end; wherein the second end is coupled to the tip; and wherein the containment member includes an opening positioned at the first end; and
a string looped around the opening of the containment member, wherein the opening at least partially closes in response to actuation of the string.

16. The device of claim 15, further including a user-actuated tightening mechanism and wherein the string is coupled to the user-actuated tightening mechanism; wherein a specimen positioned within the containment member is compressed in response to actuation of the first string by the tightening mechanism.

17. The device of claim 16, further including a handle coupled to the rod, wherein the handle includes a groove that at least partially receives the user-actuated tightening mechanism.

18. The device of claim 17, wherein the user-actuated tightening mechanism includes a faceted circumferential surface that is rotatable relative to the groove.

19. The device of claim 15, further including a sheath defining a passageway; wherein the rod is positioned at least partially within the passageway and movable with respect to the sheath; and wherein the string extends through the passageway; and wherein the opening in the containment member is oriented toward the sheath.

20. The device of claim 15, wherein the rod includes a rod passageway, and wherein the string extends through the rod passageway.

21. The device of claim 15, wherein the containment member is a first containment member and the device further includes a second containment member; wherein the first containment member is positioned at least partially within the second containment member.

22. The device of claim 21, wherein the first containment member is a mesh sleeve, and wherein the second containment member is impermeable to fluid.

23. The device of claim 21, wherein the second containment member includes a third end and a fourth end opposite the third end, wherein the second containment member includes a second opening positioned at the third end.

24. The device of claim 23, wherein a drawstring is coupled to the second opening; wherein the second opening at least partially closes in response to actuation of the drawstring.

25. The device of claim 15, wherein the string is a compression string and the device further includes an expansion string coupled to the opening of the containment member; and wherein a channel is formed in the tip and the expansion string extends through the channel.

26. The device of claim 25, wherein the opening of the containment member enlarges in response to actuation of the expansion string, and wherein a specimen positioned within the containment member is compressed in response to actuation of the compression string.

27. The device of claim 15, wherein the rod defines a longitudinal axis and the opening faces a plane that is perpendicular to the longitudinal axis.

28. A device comprising:
a first rod with a first rod end;
a second rod with a second rod end;
a containment member including a first end and a second end opposite the first end; wherein the first end is coupled to the first rod end of the first rod and the second end is coupled to the second rod end of the second rod;
wherein the containment member includes an opening positioned at the first end of the containment member;
and wherein the second rod is movable with respect to the first rod to elongate the containment member.

29. The device of claim 28, further including a sheath with a passageway, wherein the first rod and the second rod extend through the passageway.

30. The device of claim 28, wherein the second rod end moves away from the first rod end to elongate the containment member.

31. The device of claim 28, wherein the second rod is movable with respect to the first rod to adjust a cross-sectional area of the opening.

32. The device of claim 31, wherein the second rod end moves toward the first rod end to increase the cross-sectional area of the opening.

33. The device of claim 28, wherein the containment member is flexible and includes a mesh sleeve.

34. The device of claim 28, wherein the second end of the containment member is closed.

35. The device of claim 28, further comprising a string coupled to the opening; wherein the opening is movable between an open configuration and a sealed configuration in response to actuation of the string.

36. The device of claim 28, further comprising a first lock movable between an unlocked position in which the first rod is movable with respect to the second rod and a locked position in which the first rod is fixed relative to the second rod.

37. The device of claim 36, further comprising a second lock movable between an unlocked position in which the first rod is movable with respect to a sheath and a locked position in which the first rod is fixed relative to the sheath.

38. The device of claim 28, wherein the first rod includes a first handle and the second rod includes a second handle.
